(19) United States
(12) Patent Application Publication
(10) Pub. No.: US 2014/0350137 A1
(43) Pub. Date: Nov. 27, 2014

(54) BIOCOMPATIBLE X-RAY OPAQUE POLYMERS FOR MEDICAL DEVICE

(71) Applicant: ST. JUDE MEDICAL SYSTEMS AB, Uppsala (SE)

(72) Inventors: Lisa Mellin, Uppsala (SE); Karin Törne, Uppsala (SE)

(73) Assignee: ST. JUDE MEDICAL SYSTEMS AB, s-754 50 Uppsala (SE)

(21) Appl. No.: 14/366,226

(22) PCT Filed: Dec. 20, 2012
(86) PCT No.: PCT/SE2012/051463

§ 371 Date: Jun. 17, 2014

Related U.S. Application Data
(60) Provisional application No. 61/578,379, filed on Dec. 21, 2011.

(30) Foreign Application Priority Data
Dec. 21, 2011 (SE) 1151240-7

(51) Int. Cl. A61L 31/18; A61L 29/08; A61L 29/14; A61L 31/06; A61L 31/14; A61L 31/10; A61L 29/18

(52) U.S. Cl. CPC A61L 31/18; A61L 31/10; A61L 29/085; A61L 29/18; A61L 31/06; A61L 31/145; A61L 29/145; A61L 2420/06
USPC 523/113; 523/105

(57) ABSTRACT

The present application relates to a radio opaque material and a method of producing the same. The material is a hybrid material comprising two phases, an inorganic phase comprising a radio opaque substance and a polymer phase.

### FIELD OF INVENTION

The present invention relates to a radio opaque material, the material obtained by a sol-gel method, the method itself and the use of the material.

### BACKGROUND

Coronary catheterization is a minimally invasive procedure used to identify and treat coronary artery diseases. During the procedure the patient lies on a radiolucent table and an imaging camera and X-ray source move separately on opposite sides of the patient chest.

Typically a device such as pressure wire or a balloon for angioplasty is inserted into the femoral artery and guided through the main artery system to the heart. Sensor and guide wire assemblies in which a sensor is mounted at the distal end of a guide wire are known. In U.S. Pat. No. Re. 35,648, which is assigned to the present assignee, and which is incorporated by reference herein, an example of such a sensor and guide wire assembly is disclosed, where a sensor guide comprises a sensor element, an electronic unit, a signal transmitting cable connecting the sensor element to the electronic unit, a flexible tube having the cable and the sensor element disposed therein, a solid metal wire, and a coil attached to the distal end of the solid wire. The sensor element comprises a pressure sensitive device, typically a membrane, with piezoresistive elements connected in a Wheatstone bridge-type of arrangement mounted thereon. As is disclosed in, for example, U.S. Pat. No. 6,167,763, which is also assigned to the present assignee and incorporated by reference herein, the sensor element can be arranged inside a short tube (usually referred to as a sleeve or jacket), which protects the sensor element and comprises an aperture through which the pressure sensitive device is in contact with the ambient medium. U.S. Pat. No. 6,167,763 further illustrates that a first coil may be attached to the distal end of the jacket and that a similar second coil may be attached to the proximal end of the jacket. The solid metal extends through the interior of the jacket and may be provided with an enlarged diameter portion adapted for mounting of the sensor element.

After the guiding procedure is completed and the guide wire is removed pressure is applied on the entry point of the blood vessel by hand or with a closure device to prevent blood loss. This closure device may comprise one or more discs clamped together on either side of the vessel wall. The discs are usually made of biodegradable polymers that preferably degrade within 3-6 months. In U.S. Pat. No. 6,508,828, which is assigned to the present assignee and whose entire contents are incorporated herein by reference for the sealing devices and methods disclosed therein, a closure device or a sealing device is disclosed for sealing a puncture hole in a vessel wall. This sealing device comprises an inner sealing member, an outer locking member, and a retaining member. The inner sealing member is adapted to be positioned at an inner surface of the vessel wall, while the outer member is adapted to be positioned at an outer surface of the vessel wall. In use, the inner and outer members sandwich the vessel wall, and are held together by the retaining member, to thereby seal the puncture hole in the vessel wall. Other examples of sealing devices that comprise an inner member and an outer member, which are held together by an elongated retaining member, can be found in, for example, U.S. Pat. Nos. 5,593,422 and 5,620,461. Other types of medical closure devices are described in U.S. Pat. Nos. 5,531,759 and 5,282,827, whose entire contents are incorporated herein by reference for the closure devices and methods disclosed therein. These devices have an inner member in the form of an anchor member and an outer member in the form of a plug.

The catheterization procedure may need to be repeated within this timeframe and it is then important to avoid puncturing the previously placed seal when introducing the catheter again. In order to avoid that the surgeon punctures a blood vessel at the same location again during an additional coronary catheterization, surgeons today commonly use ultrasound to locate previous puncturing sites.

Another strategy would be to introduce a radio opaque substance. Hafnium, tantalum, platinum and gold are the most suitable metals for introducing radio opacity. They are all used as biomaterials; they are ductile, corrosion resistant and have a high degree of radio opacity. However simple metallic coatings on stainless steel or a polymer material are associated with some problems. Inability of the coating to follow substrate deformation may induce cracking. In addition, corrosion may cause metal ions to be released that may be toxic or cause allergic reactions. An alternative route to increase the radio opacity of organic polymers is the incorporation of iodine or iodine containing molecules. Covalent bonding of iodine to a PCL copolymer backbone with substitution rates of 25% may be achieved by a two step nucleophilic substitution reaction using lithium diisopropyl amide as a catalyst. High iodine content may however decrease the dynamic Young's modulus from 60 to 13 mPa and affect the biological degradation rate. Iodinated PCL may disclose a weight loss after 25 weeks, whereas PCL had no weight loss after 60 weeks.

### SUMMARY OF THE INVENTION

The object of the present invention is to overcome the drawbacks of the prior art and to present a method and a material that is both radio opaque and has the desired mechanical properties.

In a first aspect the present invention relates to a sol-gel method of producing a hybrid material wherein the material comprises two phases; a first and a second phase, wherein the first phase comprises an inorganic compound and the second phase comprises a biodegradable polymer, where the method comprises the steps of:
providing a first solution comprising an inorganic precursor comprising at least one metal alkoxide compound that is radio opaque, and a first solvent;
providing a second solution comprising a biodegradable polyether polymer and a second solvent miscible with the first solvent;
forming a mixture by mixing the first and the second solutions;
bringing the mixture in contact with liquid water or water in vapor phase;
letting the liquid water or water in vapor phase react with the metal alkoxide to form a sol;
letting the liquid water or water in vapor phase react further with the metal alkoxide to form a gel; and
removing the solvents to form a solid material.

In a second aspect the present invention relates to a hybrid material obtainable by a sol-gel method of producing a hybrid material comprising two phases; a first and a second phase wherein the first phase comprises an inorganic compound and the second phase comprises a polymer, where the method comprises the steps of:
providing a first solution comprising an inorganic precursor comprising at least one metal alkoxide compound that is radio opaque, and a first solvent;
providing a second solution comprising a biodegradable polyether polymer and a second solvent miscible with the first solvent;
forming a mixture by mixing the first and the second solutions;
bringing the mixture in contact with liquid water or water in vapor phase;
letting the liquid water or water in vapor phase react with the metal alkoxide to form a sol;
letting the liquid water or water in vapor phase react further with the metal alkoxide to form a gel; and
removing the solvents to form a solid material

In a third aspect the present invention relates to a hybrid material comprising two phases; a first and a second phase, wherein the first phase comprises an inorganic radio opaque compound and the second phase comprises a co-polymer of a polyether and a polyester.

In a fourth aspect the present invention relates to the use of a hybrid material as defined above for coating closure devices, catheters, guide wires, stents, sutures, light scattering material, or as membranes.

Specific embodiments of the present invention are as defined in the dependent claims that are hereby incorporated into the description.

### BRIEF DESCRIPTION OF THE FIGURES

FIG. 1 a) type I hybrid made from dispersed particles in a polymer, b) a type II hybrid made from in situ grown particles.

FIG. 2. Chemical structure of Histodenz™, used as an iodine containing initiator.

FIG. 3. The chemical reaction for end group functionalization of PCL.

FIG. 4. SEM images of a) Hyb 1.2 b) Hyb 2.1 c) Hyb 9.1 and d) Hyb 9.2.

FIG. 5. FTIR transmittance spectra of a) PCL (solid) and Hyb 4.1 (dashed) b) PEG 5 (solid) and Hyb 9.2 (dashed).

FIG. 6. FTIR transmittance spectra of a) PCL (solid), Hiz3 (thick dashed) Hiz4 (dashed) and Hiz5 (dotted) and b) Hiz3 (solid) and end group functionalized Hiz2 (dotted) Hiz3 (thick dotted) Hiz4 (dashed) and Hiz5 (thick dashed).

FIG. 7 a) suture wire dip coated with PCL b) Stainless steel wire dip coated with Hyb 1.2.

FIG. 8. a) Hyb2.1 (left) and Hyb2.2 (right) films with thickness 0.2 mm (top) and 0.1 mm (bottom) b) Discs made from iodinated polyesters. From left to right Hiz3 Hiz4 and Hiz5, the top samples are end group functionalized. A Pt-wire and a 0.5 cm thick piece of Fe are present as references.

### DETAILED DESCRIPTION OF THE INVENTION

### List of Abbreviations

### CL—Caprolactone

DCC—Dicyclohexyl carbodiimide
DLLA—DL—Lactic acid
DMAP—Dimethylamino pyridine
DSC—Differential Scanning calorimetry
EDX—Energy Dispersive X-ray spectrometry

### EGF—End Group Functionalization

### FTIR—Fourier Transform Infrared Spectroscopy

GA—Glycolic acid

### ICP-SFMS—Inductively Coupled Plasma—Sector Field Mass Spectroscopy

### IR—Infrared

LA—Lactic acid

### PCL—Polycaprolactone

PDMS—Polydimethylene siloxane
PGA—Polyglycolic acid
PLA—Polylactic acid
PTMC—Polytrimethylene carbonate

### ROP—Ring Opening Polymerization

### SEM—Scanning Electron Microscope

### TGA—Thermal Gravimetric Analysis

### THF—Tetrahydrofuran

TIBA—Triiodibenzoic acid

In the present application, the given starting point for the formation of a sol or a gel should not be seen as precise since both the sol formation and the gel formation are continuous processes, in other words the sol and gel formation could occur simultaneously.

The object of this invention is to present a method of forming a new radio opaque material with potential use as coating for guide wires, vessel closure devices and other surgery products. It is preferred that all materials should be biocompatible and the material properties should remain or at least be satisfactory even after sterilization. In one embodiment, the material should also be biodegradable.

A material consisting of two phases, one organic polymer phase and one inorganic phase, provides the possibility of combining and optimizing mechanical, optical, electrical or magnetic and of course also radio opaque properties otherwise impossible to achieve. A hybrid material of a heavy metal salt, metal particles or metal containing particles or compounds and a biocompatible elastic polymer can be both radio opaque and have the required mechanical properties. Depending on the type of interaction between the phases inorganic-organic hybrid materials can be categorized in two groups. Type I hybrid material have only week Van deer Waal or hydrogen bonds. Type II has stronger chemical bonds connecting the two phases. The materials obtained with the sol-gel method according to the present invention are of Type II.

Type I hybrids can easily be synthesized by simply mixing a fine powder of inorganic material with an organic polymer (FIG. 1a). This can be done either with a polymer or a monomer prior to polymerization. The inorganic phase can be pure metal, a ceramic material e.g. barium sulfate or zirconium dioxide or an organic-metal compound. However, due to the incompatible natures of the two phases the risk of separation is high and a leakage of particles of heavy metal/heavy metal salt into the body may be negative to the patient's health. The inorganic phase also weakens the mechanical properties of the polymer, so there is a limit to the inorganic content. Further, these hybrids also show poor adhesion to stainless steel when used as a coating material.

By using sol-gel synthesis it is possible to create highly homogeneous hybrids consisting of two nanosized interpenetrating networks; one organic and one inorganic, (FIG. 1b). Sol-gel processes are commonly used for synthesis of metal oxides where, at ambient temperature, inorganic precursors consisting of metal alkoxide(s) are hydrolyzed into a sol and then condensed to a gel.

The hydrolysis step creates reactive hydroxy groups:

M-OR+H₂O→M-OH+ROH

The hydroxy groups can then react further by two mechanisms to create an inorganic network.
a) Oxolation, the creation of an oxygen bridge

M-OH+M-OX→M-O-M+HOX (X=H or R)
b) Olation, the formation of a hydroxo bridge

M-OH+OH-M→M-(OH)₂-M

The low processing temperature allows for organic polymers to be incorporated into the synthesis. They can be added as polymers or as the corresponding monomer during or after the hydrolysis—condensation reaction. The inorganic phase will be present as nanometer sized clusters, the exact size and structure of which is determined by the rate of the different chemical reactions involved.

The rate of the hydrolysis step is determined by the reactivity of the precursor, the metal alkoxide. This in turn is determined by the electronic nature of the metal and the steric hindrance of the alkoxy groups. Silicon alkoxides are inert to hydrolysis and a catalyst is usually added. Transition metal alkoxides are more reactive and inhibitors such as strong complexing ligands (b-diketonates, acid derivatives, etc) or inorganic acids are used to control the reactivity. Another strategy is not to add water as a liquid to the hydrolysis step, but instead cure the hybrid by using the moisture in the air or to use water in vapor phase. By using the moisture in the air, or by using water in vapor phase, a slower and more controlled reaction can be obtained.

For synthesis of hybrid materials of class II, the metal alkoxide preferably comprises at least two distinct functionalities, alkoxyl group (R-OM, M=metal, bonds available to hydrolysis leading to the formation of an oxo-polymer framework) and metal to carbon links that are stable during the hydrolysis. For Si, Sn, Hg or P the C-M bond is stable. For transition metals the C-M bond is usually not stable enough and instead the link between the inorganic and organic phases can be a C—O-M bond that is stable upon hydrolysis.

Due to the ester functionalities in polyesters interacting with the metal oxides, it is possible to synthesize hybrids with polyester with or without functionalized chain ends. The organic and the inorganic networks connect via transesterfication reactions forming interactions between the carboxyl groups and the metal.

Previous reports have disclosed the dynamic mechanical properties of hybrids with PCL and tetraetoxysilane (TEOS) precursors. It was found that the number of functional end groups per polymer chain did not affect the storage modulus. The inorganic weight content and the curing temperature were found to be the two most important factors affecting the elasticity.

In one embodiment of the present invention the material should be sufficiently radio opaque for imaging, sufficiently flexible to seal a puncture, or to follow the movement of a coated substrate and degrade without formation of toxic byproducts.

The radio opaque coating should provide sufficient X-ray visibility whilst having enough flexibility and adherence to the substrate. Preferably the X-ray visibility should be similar to the visibility of existing X-ray visible devices.

The polymer could be any biocompatible polymer and the polymer could be a homopolymer or copolymer. The co-polymer could be a random, alternating, statistic or a graft co-polymer derived from two or more monomers. The polymer could be for example a polyester, polyether, polyamide, polyamine, polyacrylate, polyalkane, polyurethane, polyurethane urea, polysiloxane, polycarbonate or co-polymers thereof. Co-polymers could be, but are not limited to, for example polyester-co-ether, or polyester-co-urethane, or polyether-co-urethane, or polyester-co-polyamide or polyether-co-polyamide, or polyether-co-polyester-co-polycarbonate. A preferred co-polymer is a polyether-polyester copolymer. The ratio of polyester to polyether should preferably be between 1:20 to 20:1, for example 1:15 or less, or 1:10 or less, or 1:5 or less; or 5:1 or more, or 10:1 or more, or 15:1 or more.

The polymer could also be a multiarmed polymer or co-polymer, i.e. a polymer or a co-polymer with 2, 3, 4, 5, 6 or 7 or more arms. These structures could be obtained by using an initiator with 2 or more arms.

Examples of a polyether could be polyethylene glycol, polypropylene glycol and copolymers of polyethylene glycol and polypropylene glycol. Examples of polyesters could be polyesters based on lactic acid, glycolic acid, paradioxanone, β-butyrolactone, valerolactone, caprolactone, or a mixture of two or more of said polyesters. In order to soften or lower the glass transition temperature of a biodegradable polyester trimethylene carbonate could be added to the co-polymer, i.e. the polyester may alternatively or additionally be based on trimethylene carbonate. The molecular weight ratio between the polyether and the polyester could be between 1:100 to 1:5, for example 1:100 or more, or 1:50 or more, or 1:30 or more, or 1:20 or more, or 1:10 or more, or 1:5 or less.

The amount of glycolic acid is preferably 0-50 wt %, for example 5 wt % or more, or 10 wt % or more, or 20 wt % or more of the polyester content. The amount of lactic acid (L-lactic acid or D,L-lactic acid) is preferably 0-50 wt %, for example 5 wt % or more, or 10 wt % or more, or 20 wt % or more, but less than 50 wt %, or less than 40 wt % of the polyester content. The amount of caprolactone is preferably 0-50 wt %, for example 5 wt % or more, or 10 wt % or more, or 20 wt % or more, but less than 50 wt %, or less than 40 wt % of the polyester content. The amount of trimethylene carbonate is between 0 and 100%, for example 5 wt % or more, or 20 wt % or more, or 40 wt % or more, but less than 100 wt %, or less than 80 wt %, or less than 60 wt %. The amount of paradioxanone, β-butyrolactone and valerolactone is preferably 0-50 wt %, for example 5 wt % or more, or 10 wt % or more, or 20 wt % or more, but less than 50 wt %, or less than 40 wt % of the polyester content.

The metal part of the inorganic precursor comprises a metal selected from gold, bismuth, zirconium, platinum, tantalum, titanium and mixtures thereof, most preferably tantalum or titanium. In an embodiment, the inorganic precursor comprises a metal alkoxide selected from gold alkoxide, bismuth alkoxide, zirconium alkoxide, platinum alkoxide, tantalum alkoxide, titanium alkoxide and mixtures thereof. In a presently preferred embodiment, the inorganic precursor consists of a metal alkoxide selected from gold alkoxide, bismuth alkoxide, zirconium alkoxide, platinum alkoxide, tantalum alkoxide, titanium alkoxide and mixtures thereof. The alkoxides of the inorganic precursor according to the present invention could be, but are not limited to, C₁-C₂₀ linear or branched alkoxides, for example methoxides, ethoxides, propoxides, isopropoxide, butoxides, isobutoxides, pentoxides and isopentoxides.

The weight ratio of metal alkoxide/inorganic precursor to polymer in the method should be between 0.0001:1 to 100:1; for example 0.0001:1 or more, 0.0005:1 or more, 0.001:1 or more, 0.005:1, 0.01:1 or more, or more or 0.05:1 or more, or 0.1:1 or more; or 0.5:1 or more, or 1:1 or more, or 5:1 or more, or 10:1 or more, or 25:1 or more, or 50:1 or more, or 75:1 or more. In one embodiment the metal alkoxide to polymer weight ratio should preferably be within 0.0001:1 to 0.5:1 for example 0.0001:1 or more, or more than 0.0005:1 or more than 0.001:1, or more than 0.005:1, but less than 0.5:1, or less than 0.1:1 or less than 0.01:1.

The weight ratio of the metal oxide/first phase to the total weight of the hybrid material influences both the X-ray visibility and the mechanical properties of the hybrid material. In a preferred embodiment the weight ratio of metal oxide/first phase should be between 30 wt % and 95 wt %, for example 40 wt % or more, or 50 wt % or more, or 60 wt % or more, or 65 wt % or more or 75 wt % or more, or 95 wt % or less or 85 wt % or less, or 80 wt % or less, of the total weight of the hybrid material.

In order to be able to optimize the mechanical properties and the radio opacity, the polymer material may also contain a radio opaque substance. By introducing a radio opaque substance into the polymer material the required amount of inorganic phase to obtain the same X-ray visibility could be minimized. This radio opaque substance could be iodine, bromine or any other halogen or combinations thereof. The number of radio opaque substances per chain could be varied in order to optimize the radio opaque property and the mechanical properties.

The radio opaque substance may be introduced by using an initiator comprising said substance or by functionalizing the end groups or side groups. In one embodiment the opaque substance is preferably located at the end groups. In yet another embodiment the radio opaque substance is located in the initiator and at the end groups.

The molecular weight of the polymer should be more than 1000 g/mol, such as 2000 g/mol, preferably more than 5000 g/mol, preferably more than 10000 g/mol, preferably more than 30000 g/mol, preferably more than 50000 g/mol.

The radio opaque content in the polymer should preferably be more than 0.5 wt %, or more than 5 wt %, or more than 10 wt %, or more than 15 wt %, or more than 25 wt %, or more than 35 wt %.

One preferred embodiment comprises a PEG-co-polyester polymer having a molecular weight of at least 20000 g/mol and with a radio opaque substance content of at least 5 wt %. In this embodiment the polyester is derived from lactic acid, caprolactone and glycolic acid, and the inorganic phase consisting of tantalum oxide or titanium oxide is present in an amount of 30 wt % to 90 wt %, i.e. 30-90 wt % of the total weight of the hybrid material.

In another preferred embodiment the PEG-co-polyester has a molecular weight ratio of PEG to polyester of 1:100 to 1:5, the polyester is derived from glycolic acid, DL-lactic acid and caprolactone, and the inorganic phase consists of tantalum oxide or titanium oxide of 30 to 90 wt %, i.e. 30-90 wt % of the total weight of the hybrid material.

In another preferred embodiment the polyester part of the PEG-co-polyester comprises 30-40 wt % each of glycolic acid, DL-lactic acid and caprolactone, and the inorganic phase comprises tantalum oxide or titanium oxide at a weight ratio of 30 to 90 wt % of the total weight of the hybrid material.

In another preferred embodiment the polymer phase comprises a PEG-co-polytrimethylene carbonate polymer and the inorganic phase comprises tantalum oxide or titanium oxide at a weight ratio of 30 to 90 wt % of the total weight of the hybrid material.

In one embodiment the concentration of the metal alkoxide/inorganic precursor in the first solution is between 0.1 and 30 weight %; that is, 0.1 weight % or more, or 0.5 weight % or more, or 0.8 weight % or more, or 1 weight % or more, or 5 weight % or more, or 8 weight % or more, or 30 weight % or less, or 20 weight % or less or 15 weight % or less. In a preferred embodiment the concentration of the inorganic precursor should be in the range of 0.8-15 weight %.

In one embodiment the concentration of the biodegradable polyether-co-ester polymer in the second solution is between 5 and 45 weight %; that is, 5 weight % or more, or 10 weight % or more, or 15 weight % or more, or 45 weight % or less, or 40 weight % or less, or 30 weight % or less. In a preferred embodiment the concentration of the polymer should be in the range of 15-30 weight %.

In one embodiment the concentration of metal alkoxide/inorganic precursor in the first solution is 0.5-15 weight % and the concentration of the biodegradable polyether-co-ester polymer in the second solution is 15-30 weight %. The solvents used according to the present invention (first and second solution) should be at least partly miscible with each other, in a preferred embodiment the solvents are the same in both solutions. The solvent should be anhydrous, i.e. free from water or at least 98% free from water or preferably ≧99.5%, and could be NMP, DMSO, THF, chloroform or dichloromethane.

In order to control the hydrolysis better an acid could be added to one of the solutions. In one embodiment the acid is added to the first solution, in another embodiment the acid is added to the second solution. In yet another embodiment the acid is added to the formed mixture between the first and the second solution. Non limiting examples of acids that could be used are hydrochloric acid, sulfuric acid, phosphoric acid or acetic acid or mixtures thereof.

When adding water to form the gel, the preferred amount of water needed is at least one water molecule per alkoxide group, for example four water molecules per titanium isopropoxide and five water molecules per tantalum ethoxide. Preferably the water should be added in excess.

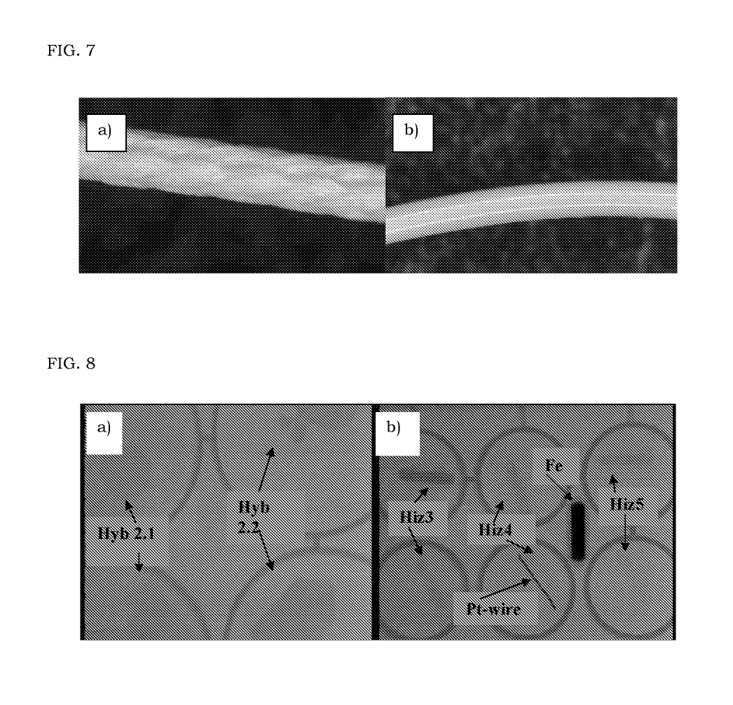

The material according to the present invention may be applied to various materials as coating. FIG. 7b discloses stainless steel dip coated with the hybrid material according to the invention. The material could also be used as a light scattering material in for example optical coherence tomography, OCT.

### Examples

### Materials

ε-caprolactone, (CL) were obtained from TCI, DL-lactic acid, (DL_LA) and Trimethylenecarbonate(TMC) were obtained from Boehringer-Ingelheim. Propanediol, tin(II) etylhexanoate and Poly(ethylene glycol) M_{w} 1000, Histodenz (5-(N-2,3-Dihydroxypropylacetamido)-2,4,6-triiodo-N,N′bis(2,3-dihydroxypropyl)isophthalamide), PDMS viscosity 750 cSt, Titanium isopropoxide, Tantalum ethoxide, Dimetylaminopyridine (DMAP), Dicyclohexylcarbodiimide (DCC), Triidobenzoic acid (TIBA) and Na₂HPO₄ were obtained from Sigma-Aldrich Co. KH₂PO₄ was obtained from Merck KGaA.

### Polymerization Reactions

Various polyesters were synthesized using ring-opening polymerizations, ROP. Tin(II)etylhexanoate was used as catalyst in all polymerization reactions.

Another set of copolymers initiated from PEG was also synthesized. In this case PEG, Mw=1000, replaced propanediol as the initiator. The catalyst and reaction conditions were the same.

### General Procedure for Ring Opening Polymerization of Polyesters

Both homopolymers and random copolymers were fabricated with target molecular weight (M_{w}) 20000 g/mol. The procedure followed standard procedure for ROP.

### General Procedure for Synthesis of Iodine Containing Polyesters

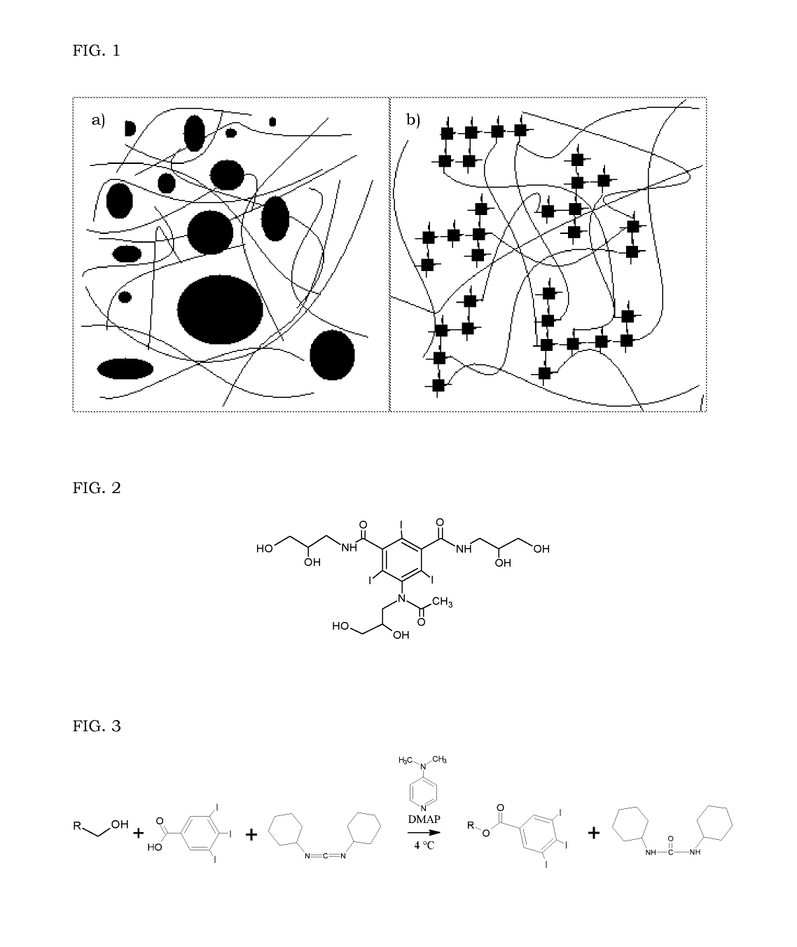

In order to incorporate iodine in the polymer back bone a non-ionic, water soluble initiator containing iodine and hydroxyl groups that can act as initiating points for polymerization was used. One example of a commercially available non-ionic, water soluble contrast agent is Histodenz™, FIG. 2. Histodenz™ contains three iodine groups and six hydroxyl groups. Polyesters of various molecular weights were synthesized according to Table 1 using Histodenz™ as initiator. Usually a higher mol % catalyst was used in order to increase the polymerization rate. Otherwise the reaction conditions were the same as for the standard ROP procedure.

### End Group Functionalization (EGF) of Iodinated Polyesters

To further increase the iodine content of polyesters initiated from Histodenz™, TIBA was coupled to the hydroxyl chain ends (FIG. 3). In a typical case 1-2 g of polyester and 8 mol % (per mol end group) DMAP was dissolved in dry THF in two separate containers. The DMAP was added to the polymer under nitrogen flow and cooled in an ice bath. In the same way as DMAP 166 mol % TIBA, and 210 mol % DCC was added. The reaction proceeded at ˜10° C. for roughly 6 hours and then at room temperature over night. The byproduct, Dicyclohexyl urea, precipitated as a white powder during the reaction. After completion it was filtered off. The THF solvent was evaporated and the polymer product was dissolved in chloroform and precipitated in methanol. Analysis of the iodine content of the resulting polymers was evaluated with ICP-SFMS.

TABLE 1
Target molecular weight and monomers ratios for polymerized
iodinated polyesters

| | | Monomer molar ratio % | | | |
|---|---|---|---|---|---|
| Polymer | Target mol. w. (g/mol) | CL | DL-LA | TMC | GA |
| Hiz1 | 3000 | 100 | | | |
| Hiz2 | 5000 | 100 | | | |
| Hiz3 | 9500 | 100 | | | 20 |
| Hiz4 | 38000 | 100 | | | |
| Hiz5 | 75000 | 100 | | | |
| Hiz6 | 10000 | | | 100 | |
| Hiz7 | 10000 | 33 | 33 | | 33 |
| Hiz8 | 10000 | 33 | | 33 | 33 |
| Hiz9 | 10000 | | 33 | 33 | 33 |

### Synthesis of Organic-Inorganic Hybrid Materials

Hybrid materials with TiO₂ or Ta₂O₅ as the inorganic phase and PDMS or the polyesters previously described, were used as the organic phase. A more detailed presentation can be seen in table 6.

### Sol-Gel Procedure for Hybrid Synthesis According to the Invention

This is a non-limiting example of the invention.

All glassware was cleaned and dried at 100° C. prior to use. One gram of a one of the polymers described above was dissolved in 4 to 5 ml dry chloroform or THF in a round bottom flask (first solution). Under nitrogen atmosphere 1-2 grams of titanium isopropoxide or tantalum ethoxide was diluted with twice as much solvent, (second solution). Optionally, 20 mol % of 37% HCl was added in order to control the hydrolysis of the metal alkoxide. The first solution was then added slowly and under stirring to the second solution. The final mixture was poured onto a Teflon sheet in a glass Petri dish and covered with a glass lid. Over night the metal alkoxide reacted with the moisture in the air and formed a sol and then gelled, and dried to a solid film. Optionally, to remove any remaining solvent and to further cure the film it was heat treated for 2 h in 100° C. in an ambient atmosphere.

### Coating of Metal Wires and Suture Threads

Both metal wires and a polylactide based degradable suture thread were dip coated with hybrid materials and polyesters. Tantalum ribbon, 30 microns thick, was spot welded onto stainless steel wires. Both bare and tantalum covered wires were dip coated in the final hybrid solution described in section above.

### Results

### Synthesized Polyesters

The appearance and the mechanical properties of polyesters synthesized from propanediol and PEG are summarized in Table 2 below. Polymers prepared from PEG were more elastic than the corresponding propanediol polymer. The white color of some of the polymers indicated a higher degree of crystallinity than the transparent ones.

TABLE 2
Properties of the synthesized polyesters

| | | Monomer (mol. ratio %) | | | | | |
|---|---|---|---|---|---|---|---|
| Polymer | Initiator | CL | DLLA | TMC | GA | Appearance | Mechanics |
| PD1 | Prop. diol | 100 | | | | white powder | hard fragile |
| PD2 | Prop. diol | | 100 | | | white | soft fragile |
| PD3 | Prop. diol | | | 100 | | transparent | very elastic |
| PD4 | Prop. diol | | | 50 | 50 | white | soft waxy |
| PD5 | Prop. diol | 33 | 33 | | 33 | transparent | soft elastic |
| PD6 | Prop. diol | 33 | | 33 | 33 | opaque | soft waxy |
| PEG1 | PEG | 100 | | | | white powder | hard durable |
| PEG2 | PEG | | 100 | | | transparent | hard fragile |
| PEG3 | PEG | | | 100 | | transparent | soft elastic |
| PEG4 | PEG | 33 | | 33 | 33 | white | soft brittle |
| PEG5 | PEG | 33 | 33 | | 33 | transparent | soft elastic |

Prop. diol = propane diol

TABLE 3
Properties of synthesized iodinated polyesters after EGF.

| | After functionalization | | | | |
|---|---|---|---|---|---|
| | | | Iodine | Iodine | |
| Polymer | Appearance | Mechanics | wt % cal* | wt % exp** | Δ H |
| Hiz2 | brown | soft weak | 45.78 | — | — |
| Hiz3 | light brown | hard fragile | 25.76 | 17.5 | 43 |
| Hiz4 | light brown | hard fragile | 6.86 | 6.69 | 50 |
| Hiz5 | white | soft weak | 3.5 | 4.06 | 47 |
| Hiz6 | light brown | soft weak | 24.5 | — | — |
| Hiz7 | light brown | soft elastic | 24.5 | 18.2 | — |
| Hiz8 | light brown | sot elastic | 24.5 | — | — |
| Hiz9 | light brown | soft tough | 24.5 | — | — |

### Iodinated Polyesters

Some properties of polyesters initiated by Histodenz™ are presented in Table 2. Table 3 features the same polymers after EGF. The iodine induces a yellow/brown color, the intensity of which increases with the amount of iodine. Histodenz™ have six potential initiation sites and thus produces multiarmed polymers. The multiarmed structure entails slightly more flexible mechanical properties than the analogous linear polymer. The EGF increased this difference further. It is likely that the bulky TIBA end group molecule hinders crystallization and therefore changes the mechanical properties. Further evidence of this is given by the DSC measurements.

TABLE 4
Properties of synthesized iodinated polyesters

| | Before functionalization | | | | |
|---|---|---|---|---|---|
| | | | Iodine | Iodine | |
| Polymer | Appearance | Mechanics | wt % cal* | wt % exp** | Δ H |
| Hiz1 | yellow | Soft | 9.97 | — | — |
| Hiz2 | light yellow | hard fragile | 6.54 | — | 57 |
| Hiz3 | white | hard fragile | 3.68 | 3.23 | 61 |
| Hiz4 | white | hard fragile | 0.98 | 0.93 | 58 |
| Hiz5 | white | soft weak | 0.5 | 0.53 | 55 |
| Hiz6 | yellow | very soft | 3.5 | — | — |
| Hiz7 | white | soft elastic | 3.5 | 2.73 | — |
| Hiz8 | light yellow | soft glutinous | 3.5 | — | — |
| Hiz9 | white | soft tough | 3.5 | — | — |

### Hybrid Materials

Clusters of nanometer size are too small to scatter visible light; therefore the nanocomposite hybrids were usually transparent. The PEG block copolymer hybrids however had a white color. This may be an indication that the hydrophilic PEG and the metal oxide form larger clusters.

The effect of the inorganic phase on the mechanical properties of the polymers was detrimental. Most hybrids were hard and fragile. The only polymers that gave soft flexible hybrids were PDMS, PEG 3 and PEG 5. The PDMS hybrids were flexible but not very durable when subjected to strain. The PEG hybrids were tougher and had more rubbery properties, see Table 5.

TABLE 5
Properties of the synthesized hybrid materials

| | | Inorg. | Org. | | | | |
|---|---|---|---|---|---|---|---|
| Hybrid | Ratio* | Prec. | Prec. | Solv. | Additives | Color | Mechanics |
| Hyb 1.1 | 25 | TiO₂ | PDMS | THF | HCl | transp. | soft flexible |
| Hyb 1.2 | 50 | | | | | white | soft flexible |
| Hyb 1.3 | 75 | | | | | white | soft flexible |
| Hyb 2.1 | 25 | Ta(EtOH)₅ | | | | transp. | soft flexible |
| Hyb 2.2 | 50 | | | | | transp. | soft flexible |
| Hyb 2.3 | 75 | | | | | white | soft flexible |
| Hyb 3.1 | 25 | TiO₂ | PD 1 | | | transp.** | hard fragile |
| Hyb3.2 | 50 | | | | | transp. | hard fragile |
| Hyb 3.3 | 75 | | | | | transp.** | hard fragile |
| Hyb 4.1 | 25 | Ta(EtOH)₅ | | | | transp.** | hard fragile |
| Hyb 4.2 | 50 | | | | | transp.** | hard fragile |
| Hyb 4.3 | 75 | | | | | transp.** | hard fragile |
| Hyb 5 | 50 | Ta(EtOH)₅ | PD 4 | CHCl₃ | | white | hard fragile |
| Hyb 6.1 | 25 | | PD 5 | | | white | soft fragile |
| Hyb 6.2 | 50 | | | | | transp. | hard fragile |
| Hyb 7 | 50 | | PEG 3 | | HCl | white | soft elastic |
| Hyb 8 | 50 | | PEG 4 | | | white | hard fragile |
| Hyb 9.1 | 25 | | PEG 5 | | | white | soft elastic |
| Hyb 9.2 | 50 | | | | | white | soft elastic |

*The molar ratio of inorganic to organic precursor
**These hybrids were transparent at first but changed to white after they had been stored at RT for a few weeks
Transp. = transparent

### SEM Images

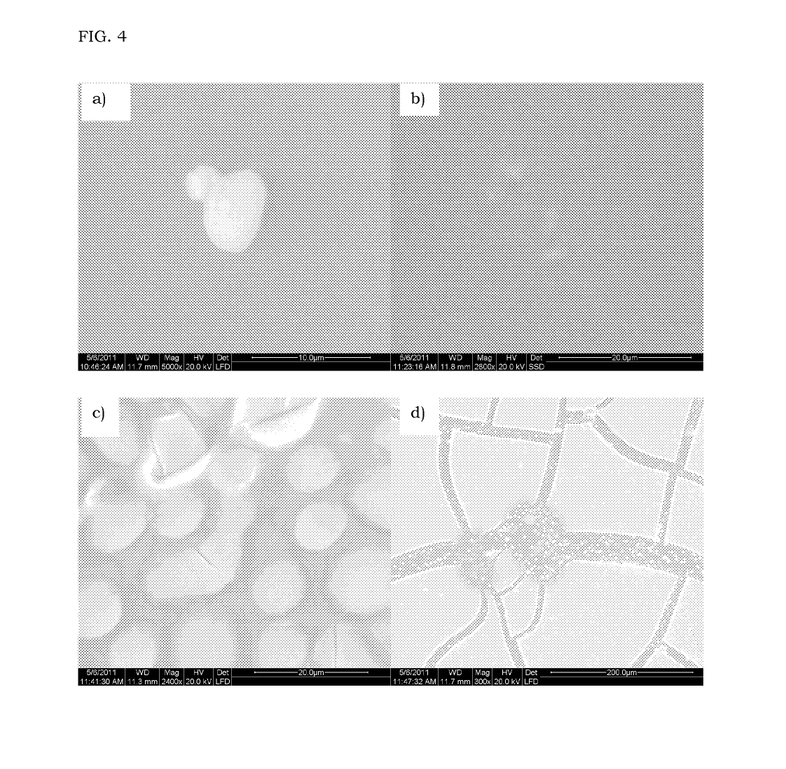

The SEM images of Hyb 1.2 and 2.1 (FIGS. 4a and 4b) showed a smooth homogeneous surface with the exception of a few adsorbed foreign particles. Hyb 9.1 and 9.2 however showed extensive phase separation (FIGS. 4c and 4d). The EDX analysis showed elevated metal content in Hyb 9.1 and 9.2 compared to the theoretical values supported by the TGA results. Hyb 9.1 had a surface metal content of 20 wt % compared the theoretical value of 13 wt %. The bright areas in Hyb 9.2 had 57 wt % metal content and the dark areas 37 wt %. It is possible that the hydrophilic PEG areas of the polymer attract more of the metal oxide forming the white areas.

### FTIR-Spectra

### Hybrid Materials

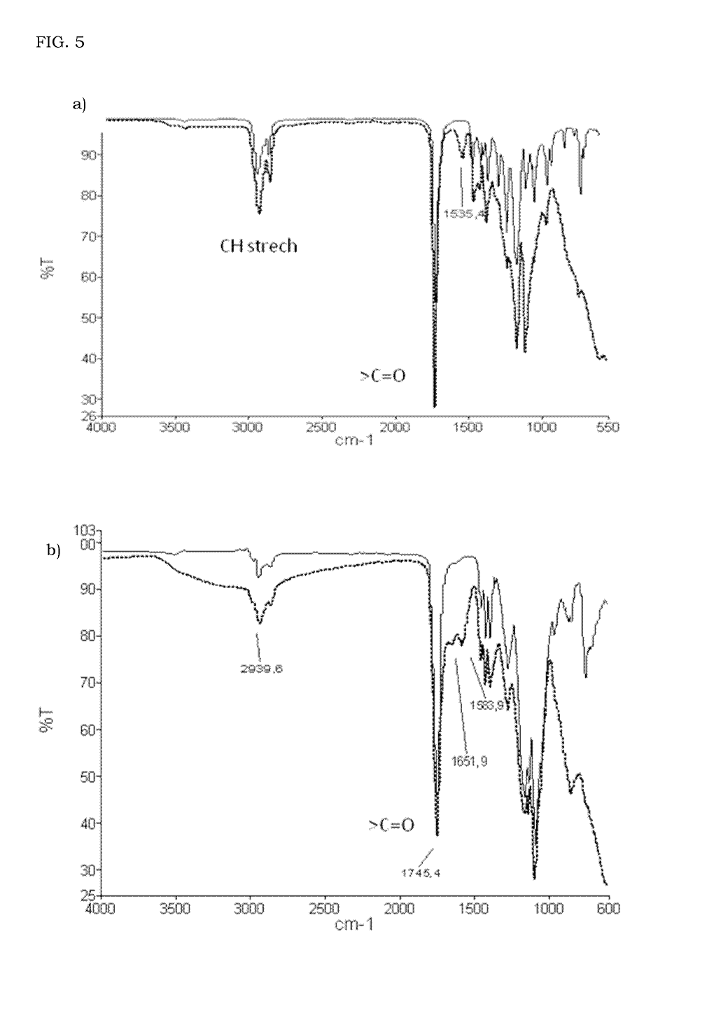

The spectra of the ester polymers (FIG. 5a) were dominated by two areas of peaks. First at 2950-2850 cm⁻¹ peaks from the C—H stretch of the hydrocarbon chain were observed. At lower wave numbers the sharp bands between 1036 and 1600 cm⁻¹ were attributed to the —CH₂— deformation. The highest peak of the spectra at 1721 cm⁻¹ came from the carbonyls (>C═O). It is these bonds that interact with the inorganic oxides, and due to the electron donating nature of the metals new peaks appeared just below 1721 cm⁻¹. The Hyb4.1 had one additional peak at 1532 cm⁻¹ which in the literature is assigned to a bidentate bridging structure.

The PEG-co-polyester tantalum oxide hybrids showed two shoulders on the carbonyl peak at 1652 and 1580 cm⁻¹ (FIG. 5b). These shoulders indicate monodentate and bidentate chelating interactions between the inorganic and organic phase. The PEG hybrids spectra also had a broad peak at 3500-1700 cm⁻¹. One possibility is that these hybrids absorb more water than their organic precursors due to the inorganic phase. However after the hybrids were dried in vacuum for 3 days the peak was unchanged.

### Iodinated Polyesters

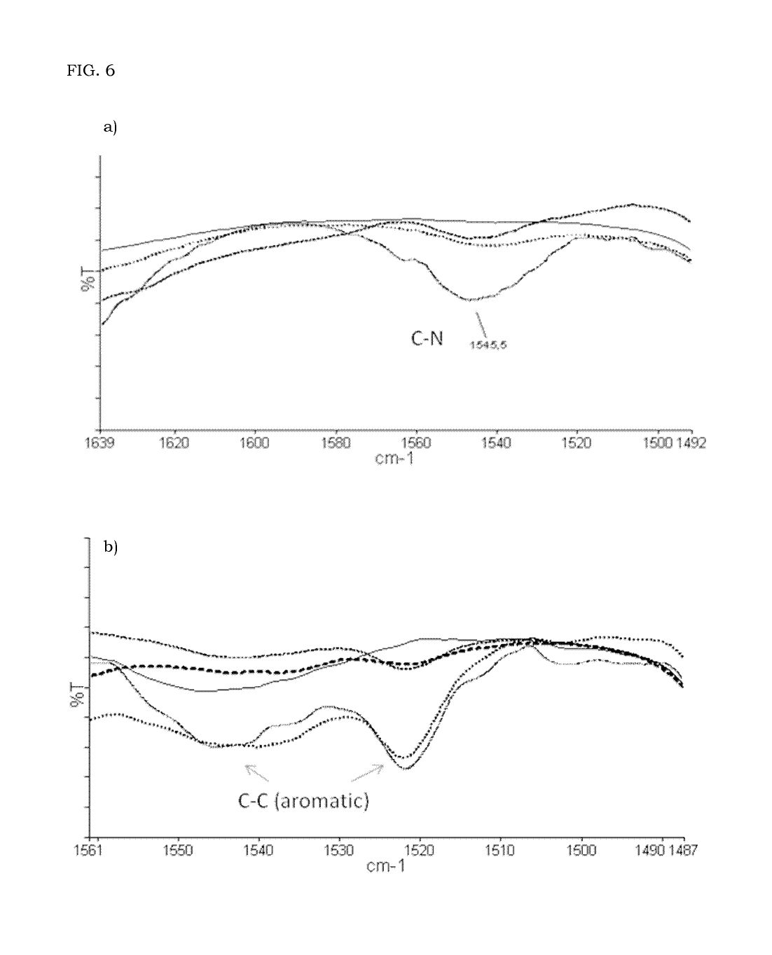

Compared to the polyesters initiated by propanediol, the Histodenz™ initiated polyesters showed additional peaks due to C—N stretching at 1627 and 1545 cm⁻¹ (FIG. 6a). After EGF with TIBA, peaks due to aromatic C—C stretching appeared at 1545 and 1521 cm⁻¹ (FIG. 6b) and at 3000-3100 cm⁻¹ due to aromatic C—H stretching (spectrum not shown). The intensity of these peaks grew stronger by increasing the Histodenz™ and/or TIBA content

### X-ray Visibility

### Hybrids

The degree of X-ray opacity of hybrid films corresponded to the amount and kind of inorganic content regardless of the kind of polymer used. Hybrids that had a titanium oxide inorganic phase absorbed less radiation than the tantalum oxide hybrids (FIG. 8a and b).

### Iodinated Polyesters

These polymers had a lower X-ray absorption prior to EGF (FIG. 8b bottom). Only the polymers with very low molecular weight had some visibility. After the iodine content was increased by EGF the visibility increased as well (FIG. 8b top).

1. A sol-gel method of producing a hybrid material comprising two phases; a first and a second phase wherein the first phase comprises an inorganic compound and the second phase comprises a biodegradable polymer, where the method comprises the steps of:
providing a first solution comprising an inorganic precursor comprising at least one metal alkoxide compound that is radio opaque, and a first solvent;
providing a second solution comprising a biodegradable polyether polymer and a second solvent miscible with the first solvent;
forming a mixture by mixing the first and the second solutions;
bringing the mixture in contact with liquid water or water in vapor phase;
letting the liquid water or water in vapor phase react with the metal alkoxide to form a sol;
letting the liquid water or water in vapor phase react further with the metal alkoxide to form a gel; and
removing the solvents to form a solid material.

2. The method of claim 1, wherein the biodegradable polymer is a co-polymer of a polyether and a polyester.

3. The method of claim 1, wherein the polyether is based on polyethylene glycol, polypropylene glycol or co-polymers of polyethylene glycol and polypropylene glycol.

4. The method of claim 2, wherein the co-polymer is a PEG-co-polyester polymer.

5. The method of claim 2, wherein the polyester is based on lactic acid, glycolic acid, caprolactone, trimethylene carbonate, paradioxanone, β-butyrolactone, valerolactone, or a mixture of two or more of said polyesters.

6. The method of claim 1, wherein the polymer is a multiarmed polymer with 2, 3, 4, 5, 6 or 7 or more arms.

7. The method of claim 1, wherein the molecular weight of the polymer is more than 1000 g/mol, such as 2000 g/mol, preferably more than 5000 g/mol.

8. The method of claim 1, wherein the inorganic precursor comprises tantalum alkoxide, titanium alkoxide, gold alkoxide, bismuth alkoxide or zirconium alkoxide.

9. The method of claim 1, wherein the weight ratio of inorganic precursor to polymer is at least 1:1, or preferably 5:1, or even more preferred 10:1.

10. The method of claim 1, wherein the polymer further contains a radio opaque substance.

11. The method of claim 10, wherein the content of the radio opaque substance in the polymer is more than 0.5 wt %, such as more than 5 wt %, or more than 10 wt %, or more than 15 wt %, or more than 25 wt %, or more than 35 wt %.

12. The method of claim 10, wherein the radio opaque substance contained in the co-polymer is iodine, bromine or any other halogen or combinations thereof.

13. The method of claim 12, wherein the radio opaque substance is iodine, which is incorporated in the polymer backbone by initiation of ring-opening polymerization by an initiator containing iodine and hydroxyl groups, and optionally further by end group functionalization of the iodinated polyester with triiodobenzonic acid.

14. The method of claim 1, wherein the concentration of inorganic precursor in the first solution is 0.8-15 weight % and the concentration of the biodegradable polyether polymer in the second solution is 15-30 weight %.

15. The method of claim 1, wherein an acid is added to the first or the second solution or to the mixture of the first and the second solution.

16. The method of claim 1, wherein the weight ratio of metal oxide is between 30 wt % and 95 wt % of the total weight of the material.

17. A hybrid material obtainable by a sol-gel method of producing a hybrid material comprising two phases; a first and a second phase wherein the first phase comprises an inorganic compound and the second phase comprises a biodegradable polymer, where the method comprises the steps of:
providing a first solution comprising an inorganic precursor comprising at least one metal alkoxide compound that is radio opaque, and a first solvent;
providing a second solution comprising a biodegradable polyether polymer and a second solvent miscible with the first solvent;
forming a mixture by mixing the first and the second solutions;
bringing the mixture in contact with liquid water or water in vapor phase;
letting the liquid water or water in vapor phase react with the metal alkoxide to form a sol;
letting the liquid water or water in vapor phase react further with the metal alkoxide to form a gel; and
removing the solvents to form a solid material.

18. A hybrid material obtainable by a sol-gel method of producing a hybrid material according to claim 1.

19. A hybrid material comprising two phases; a first and a second phase wherein the first phase comprises an inorganic radio opaque compound and the second phase comprises a biodegradable co-polymer of a polyether and a polyester.

20. The hybrid material of claim 19, wherein the first phase comprises a radio opaque metal oxide selected from tantalum oxide, titanium oxide, bismuth oxide, zirconium oxide or gold oxide.

21. The hybrid material of claim 20, wherein the metal oxide has a weight ratio of 40 to 90 wt % of the total weight of the hybrid material.

22. The hybrid material of claim 19, wherein the polyester is based on lactic acid, glycolic acid, caprolactone, trimethylene carbonate, paradioxanone, β-butyrolactone, valerolactone, or a mixture of two or more of said polyesters.

23. The hybrid material of claim 19, wherein the co-polymer comprises a radio opaque substance.

24. The hybrid material of claim 23, wherein the content of the radio opaque substance in the polymer is more than 0.5 wt %, such as more than 5 wt %, or more than 10 wt %, or more than 15 wt %, or more than 25 wt %, or more than 35 wt %.

25. The hybrid material of claim 23, wherein the radio opaque substance contained in the co-polymer is iodine, bromine or any other halogen or combinations thereof.

26. The hybrid material of claim 19, wherein the co-polymer is a multiarmed co-polymer with 2, 3, 4, 5, 6 or 7 or more arms.

27. The hybrid material of claim 19, wherein the molecular weight of the co-polymer is more than 1000 g/mol, such as 2000 g/mol, preferably more than 5000 g/mol.

28. The hybrid material of claim 19, wherein the material comprises a PEG-co-polyester polymer having a molecular weight of at least 20000 g/mol and with a radio opaque substance content of at least 5 wt % and where the polyester is derived from lactic acid, caprolactone and glycolic acid and the inorganic phase comprising tantalum oxide or titanium oxide is present in a molar ratio of 25:1 to 75:1 to the polymer.

29. The hybrid material of claim 19, wherein the material comprises a PEG-co-polyester in which the polyester part comprises 33 wt % each of glycolic acid, DL-lactic acid and caprolactone and the inorganic phase consists of tantalum oxide or titanium oxide having a weight ratio of 60 to 90 wt % of the total weight of the material.

30. Use of a hybrid material of claim 19 for coating closure devices, catheters, guide wires, stents, sutures, light scattering material, or as a membrane.
